(19) United States
(12) Patent Application Publication
(10) Pub. No.: US 2023/0042678 A1
(43) Pub. Date: Feb. 9, 2023

(54) METHOD FOR THE AUTOMATED PRODUCTION OF INDIVIDUALIZED TABLETS, AND TABLET PRESS FOR THE AUTOMATED PRODUCTION OF INDIVIDUALIZED TABLETS

(71) Applicant: PrivMed X AB, Akersberga (SE)

(72) Inventors: Ingo KRAUSE, Akersberga (SE); Rene MANIGK, Ottenbach (DE)

(73) Assignee: PrivMed X AB, Akersberga (SE)

(21) Appl. No.: 17/970,837

(22) Filed: Oct. 21, 2022

Related U.S. Application Data
(63) Continuation of application No. PCT/EP2021/053607, filed on Feb. 15, 2021.

(30) Foreign Application Priority Data
Apr. 22, 2020 (EP) 20170820.3

(51) Int. Cl. A61K 9/20; A61J 3/10; B30B 11/08; B30B 15/06; B30B 15/30

(52) U.S. Cl. CPC A61K 9/2095; A61J 3/10; B30B 11/08; B30B 15/065; B30B 15/302

(57) ABSTRACT

A method for producing tablets with an active substance including inputting a target number of tablets to produce and a target mass indicating the quantity of active substance per tablet, transmitting the target number and target mass to a controller configured to control a dosing device, a weighing device, extendable upper and lower punches, filling a quantity of the active substance from a container into an opening of a die. While filling, a pressure face of the lower punch closes the opening, and the amount of the active substance is measured by the weighing device on which the lower punch and the die are placed while filling. The method further including pressing the active substance, releasing a pressed tablet from the die, and repeating the repeating the process until the target number is reached.

### CROSS-REFERENCE TO RELATED APPLICATION

The present application is a continuation of PCT/EP2021/053607 filed on Feb. 15, 2021, which is based upon and claims the benefit to EP 20170820.3 filed on Apr. 22, 2020, the entire contents of each of which are incorporated herein by reference.

### BACKGROUND

### Field

The present disclosure relates to a method for automatically producing a specifiable number of individualized tablets with a specifiable quantity of a first active substance by a tablet press.

The present disclosure also relates to a tablet press for automatically producing a specifiable number of individualized tablets with a specifiable quantity of a first active substance.

### Prior Art

Tablets play an indispensable role in medical and non-medical fields today, from the administration of medications to dietary supplements.

To produce tablets, a die with a central opening is frequently used, into which the active substance from which the tablet is to be produced is filled. By a lower punch and an upper punch, the active substance in the central opening is pressed in order to form a tablet.

To meet the great need for tablets, the tablets are frequently produced in standardized sizes with a defined quantity of active substance. For this purpose, rotary presses, for example, are used which comprise up to a hundred dies and punch pairs, which are driven in a rotating and synchronized manner by a rotating plate. Such rotary presses can produce up to 1.6 million tablets per hour.

However, especially in medical applications, the active substance quantities to be administered are dependent on the individual patients, the absorption capacity of the active substance, and the current state of health of these patients. Especially, but not only, children and older people require a specially tailored adjustment of the dose.

Since common medical preparations of active substances are usually only available in a number of standard doses, doctors and pharmacists almost always have to select a dose that is too high, since a dose that is too low would not have the desired treatment effect. However, doses that are too high unnecessarily strain the patients' organ system and lead to damage in the long term. Splitting tablets into smaller pieces by a knife is not feasible or practical for patients.

In addition, tablets are frequently sold in tablet packages with a specified number of tablets. This leads to waste of raw pharmaceutical materials and extra tablets that are not needed by the patient and are thrown away.

### SUMMARY

An object is to simplify dosing and intake of active substances administered in tablet form that is individualized for patients.

Such object can be achieved by a method for automatically producing a specifiable number of individualized tablets with a specifiable quantity of a first active substance by a tablet press, which can be a compact tablet press, the method comprising:

inputting a target number which indicates the number of tablets to be produced and a target mass which indicates the quantity of the first active substance per tablet into an input device,

transmitting the target number and the target mass from the input device to a controller of the tablet press, wherein the controller is configured to control a first dosing device, a weighing device, a vertically extendable upper punch, and a vertically extendable lower punch,

filling a quantity of the first active substance which corresponds to the target mass from a first material container into a central opening of a die by the first dosing device, wherein while filling, a pressure face of the lower punch closes the central opening, and the amount of the first active substance filled into the central opening is measured by the weighing device on which the lower punch and the die are placed while filling,

pressing the first active substance by extending a pressure face of the upper punch into the central opening,

releasing a pressed tablet from the die by extending the pressure face of the lower punch into the central opening,

repeating the adding of the quantity of the first active substance, pressing of the first active substance, and releasing of the pressed tablet until the number of pressed tablets corresponds to the target number.

A tablet container can be arranged in a receiving chamber of the tablet press before the method and, after the release of the pressed tablet, this tablet can be transferred into the tablet container by a transfer device controlled by the controller.

Such method can provide the patient with a specifiable target number of individualized tablets with a specifiable target mass of the first active substance. The target mass can be defined, for example, by a doctor or a pharmacist, taking into account the state of health and the physical characteristics of the patient, such as, for example, his body weight. Since the active substance contained in the tablet corresponds exactly to the target mass, splitting the tablet is unnecessary. In this manner, doses that are too high can be advantageously avoided and taking the tablets is made easier for the patient.

By arranging the lower punch and the die on the weighing device, the material of the first active substance filled into the central opening can be precisely and reliably measured while filling.

The target number can also be defined, for example, by the doctor or the pharmacist. In doing so, how quickly the state of health of the patient changes and how often an adjustment of the dose is required can also be taken into account. The target number can be chosen such that the produced tablets are sufficient for treating the patient until the next diagnosis without excess tablets being produced. Wasting tablets by issuing standardized tablet packages that contain more than the required tablets can be advantageously avoided.

The tablet press can be compact. This can be achieved in that the tablet press can comprise only a single upper punch and/or a single lower punch. The tablet press can be located, for example, in a doctor's office, a pharmacy, or at the patient's home. The tablets can be produced immediately after the determination of the target mass and target number in the doctor's office, in the pharmacy, or by the patient himself at home. A decentralized method for producing individualized tablets outside of industrial production facilities can thus be provided. Since the number of the individualized tablets required by a patient is relatively small, high production speed is not necessary for their production. Instead, it is advantageously provided that a plurality of tablet presses in a plurality of doctor's offices, pharmacies, or patient dwellings meet the patients' needs for individualized tablets.

The first material container can be releasably fixed to a first dosing opening, such as, of a press chamber, of the tablet press. This can enable the first material container to be advantageously quickly changed, for example when tablets with a different active substance are to be produced. The first dosing device can be a component of the first material container and can form a unit therewith. The first active substance can be either a single active substance or a mixture of different active substances. In particular, the first dosing device can be a closable funnel or a container with a closable opening on the lower side or a container with a funnel or a container with a pipette.

The upper punch and/or the lower punch can be vertically extendable. The outer circumference of the pressure face of the upper punch and/or the pressure face of the lower punch can correspond to an inner circumference of the central opening in the die. The pressure face of the lower punch can close the central opening from below in that the pressure face of the lower punch can be pushed into the central opening from below. This can create a trough, the walls of which are formed by the central opening and the bottom of which is formed by the pressure face of the lower punch. To press the first active substance, the presto sure face of the upper punch can be extended vertically from above into the central opening while pressing. To release the pressed tablet, the pressure face of the upper punch can be moved back upward, and the pressure face of the lower punch can be extended upward until it is flush with the upper edge of the die.

The input device can comprise input means for inputting the target mass and the target number and can be a touchscreen. The input device can also be configured to display the input target number and/or the input target mass and/or additional information, such as patient information and/or information about the first active substance, on a data display. The input device can further comprise an input means with which the production of the tablets is started. The input device can comprise a security access system. For example, the input device can be secured by an access code or can comprise a fingerprint scanner. According to one embodiment, the input device can be a fixed component of the tablet press. According to another embodiment, the input device can be an external unit which transmits the target number and the target mass to the controller. According to another embodiment, the input device can be an interface of the tablet press by which the target number and target mass can be transmitted from an external unit to the controller.

The target number and the target mass can be coded in an identifier, such as a barcode, which can be printed onto a prescription which is issued to the patient. The identifier can be read by an image capture device of the input device, such as a camera, and transmitted to the controller, wherein the controller can determine the target number and the target mass from the identifier.

According to one embodiment, the press chamber can be removably fixed in the tablet press. The press chamber together with the lower punch arranged therein and the die arranged therein can be configured as an exchangeable press chamber insert.

The controller can be a computer configured to read out the target mass and target number input into the input device and actuate the first dosing device, the weighing device, the upper punch, the lower punch, and the transfer device.

The weighing device can be a weight sensor. For example, the weight sensor can be a high precision weight sensor, for example a piezoelectric load cell or an electromagnetic force compensation type sensor. The weight sensor may also be a strain gauge.

The transfer device can comprise a pushing device, by which the produced tablet is pushed down by the pressure face of the lower punch. The transfer device can further comprise a channel that opens above a tablet container arranged in the receiving chamber. The tablets pushed down by the pressure face of the lower punch fall through the channel into the tablet container Immediately after the transfer of the pressed tablet, the next tablet is produced until the target number is reached.

According to one embodiment, the controller can calculate a filling volume and thus a target circumference, taking into account the target mass, and if necessary a bulk density, of the first active substance, select a target die from at least two dies arranged in the tablet press with different inner circumferences, the inner circumference of the target die's central opening corresponding to the target circumference, and actuate a first positioning device such that the target die is arranged above the lower punch, wherein the first positioning device can be a rail device on which the at least two dies arranged in the tablet press are arranged, such as being fixed. The rail device may include a linear actuator configured to move the rail along the horizontal direction.

In another embodiment, the first positioning device may be a disc device. The disc device may include a disc and an actuator configured to rotate the disc. The dies may be arranged in recesses of the disc.

The size of the pressed tablets can be varied in this manner. To do this, the controller can automatically determine a filling volume and thus an appropriate target circumference using the target mass, and if necessary, the bulk density, of the first active substance, select an appropriate target die, and arrange it above the lower punch. Manually changing the die is thus unnecessary. The variability of the size of the pressed tablets can enable individualized tablets with very different active substance quantities to be produced.

At least three dies with different inner circumferences of their central openings can be fixed to the first positioning device.

The outer circumference of the pressure face of the upper punch for pressing the first active substance can be adapted to the inner circumference of the die in that a number of vertically telescoping upper punch shafts of the upper punch arranged inside each other are extended downwards, wherein the outer circumference of the outer extended upper punch shaft can correspond to the inner circumference of the die.

By adapting the outer circumference of the pressure face of the upper punch to the inner circumference of the die, replacing the upper punch when using dies with different inner circumferences advantageously becomes unnecessary. The outer circumference of the pressure face of the upper punch can be adapted by extending the upper punch shafts. The pressure face of the upper punch can correspond to the entirety of the pressure faces of the extended upper punch shafts. The pressure faces of the upper punch shafts can be formed such that the pressure face of the upper punch is always continuous.

For each die arranged on the first positioning device, the upper punch can comprise an associated upper punch shaft, the cross-sectional shape of which can correspond to the cross-sectional shape of the central opening of one of the dies. The upper punch shaft associated with the central opening and all upper punch shafts arranged farther inwards can always be extended to press the first active substance. The upper punch shafts can be substantially arranged inside each other basically without gaps. In this manner, the pressure face of the upper punch can always be free of grooves, such that tablets with a smooth surface are produced.

The outer circumference of the pressure face of the lower punch can be adapted to the inner circumference of the die before the filling of the quantity of the first active substance in that a number of vertically telescoping lower punch shafts of the lower punch arranged inside each other can be extended upwards, wherein the outer circumference of the outer extended lower punch shaft can correspond to the inner circumference of the die.

By adapting the outer circumference of the pressure face of the lower punch to the inner circumference of the dies, replacing the lower punch when using dies with different inner circumferences advantageously also becomes unnecessary. The outer circumference of the pressure face of the lower punch can be adapted to the inner circumference of the dies before the pressure face of the lower punch is extended into the central opening from below in order to close it from below.

The lower punch shafts can be made as a mirror image to the upper punch shafts such that the advantages and characteristics described with regard to the upper punch shafts also apply to the lower punch shafts.

According to one embodiment, the lower punch and the die can form a unit, wherein the lower punch and die can be movable relative to each other in a vertical direction. The lower punch and the die can be coupled by a spring. The spring can draw the lower punch back into the starting position by its spring force after the lower punch is extended.

The mass of the first active substance filled into the central opening can be measured by placing the die and the lower punch on the weighing device before filling, such as by at least one lifting device, wherein the weighing device measures the total mass acting on it while filling, which can comprise the mass of the die, the mass of the lower punch, and the mass of the first active substance filled into the central opening, wherein the die and the lower punch can be lifted after filling and before pressing the first active substance, such as by the at least one lifting device.

The lifting device can include a mechanism with a platform, spindles and an actuator. The actuator may be a motor. The die and the lower punch may be arranged on the platform. The platform may be arranged on the spindles. The actuator may be configured to extend or retract the spindles. In doing so, the platform, the die and the lower punch are moved up and down. The platform can comprise a central recess. The weighing device is arranged in such a way, that it is below the platform when the spindles are maximally extended. When the platform is moved down, the weighing device enters the recess of the platform. The die and the lower punch can be arranged on the platform in such a way, that they are placed on top of the weighting device when the platform is lowered. When the platform is lowered further, the die and the lower punch stay on the weighing device and lose contact to the platform.

After placing the die and the lower punch and before filling, the total mass can be measured in order to obtain a measured value for the unfilled state. This measured value can be subtracted from the total mass while filling in order to calculate the mass of the first active substance filled into the central opening. Filling can be ended when the mass of the first active substance filled into the central opening corresponds to the target mass. To control the filling process, the measured data of the total mass recorded by the weighing device can be transmitted to the controller. From the measured data of the total mass, said controller can calculate the current mass of the first active substance filled into the central opening. Once the target mass is achieved, it can control the dosing device of the first material container to end filling. The controller can couple the weighing device to the first dosing device by an algorithm.

After lifting, the die and the lower punch can no longer rest on the weighing device so that the weighing device is not damaged while pressing. The lifting and lowering can be performed by at least one lifting device. The lifting device can be configured to move the die and the lower punch in a vertical direction. The lower punch and/or the at least one die can rest on a platform that can be moved by the at least one lifting device in a vertical direction. After the lower punch and the die have been placed on the weighing device, the platform can be moved downward until the lower punch and the die rest exclusively on the weighing device while filling, and no longer on the platform.

The pressure acting on the lower punch and/or the upper punch can be measured at the at least one lifting device, such as by a pressure measuring device.

In this way, the acting pressure can be measured during the pressing process. The pressure measuring device can be a force transducer in the sense of a pressure load cell. For example, the pressure measuring device is a piezoelectric load cell, a pneumatic load cell or a hydraulic load cell. By such pressure measuring device, the pressure during the production of the tablets can advantageously be monitored, a tablet hardness and/or break resistance of the tablets can be controlled, and damage to the punches and/or dies can be prevented.

The first dosing device can be arranged vertically above the central opening by a second positioning device, such as a rail device that can be moved in a horizontal direction, before adding the quantity of the first active substance, wherein the upper punch can be arranged vertically above the central opening by the second positioning device after adding the quantity of the first active substance and before pressing the first active substance, wherein the first dosing device can also be arranged, such as by being fixed, on the second positioning device so as to be movable in the vertical direction.

The second positioning device can advantageously simplify adding the first active substance. By arranging the first dosing device above the central opening, the first active substance can be added from above directly and without loss. After adding the first active substance, the first dosing device can be moved to the side such that the upper punch is arranged above the central opening.

The first dosing device can be moved in a horizontal direction by the second positioning device while adding the first active substance. The first dosing device can be moved repeatedly in a horizontal direction from left to right in the die by the second positioning device while filling. This can be advantageous while filling large and/or oval dies in order to evenly distribute the active substance.

A second dosing device can be moved in a horizontal direction by the second positioning place. In this way, it is possible to produce a tablet with two adjacent layers. For this purpose, the first dosing device can be moved over a first corner of the die, and the second dosing device can be moved over a second corner of the die, the first corner can be filled with the first active substance by the first dosing device, and the second corner can be filled with a second active substance by the second dosing device, and then a two-layer tablet with adjacent layers can be pressed. The second dosing device may be configured similarly as the first dosing device.

A rail device offers a reliable and compact positioning device by which the first dosing device and the upper die can be moved in the horizontal direction. In that the first dosing device can be also movable in the vertical direction, the first dosing device can be lowered after positioning the first dosing device above the central opening enough for the lower edge of the first positioning device to be located below the upper edge of the at least one die. In other words, the first dosing device can be lowered into the central opening. In this manner, spillage of the active substance is prevented while filling so that the entire amount of the active substance enters the central opening. The rail device may include a rail and a linear actuator configured to move the rail along the horizontal direction. For example, the first dosing device and the upper punch can be fixed to a lower side of the rail. In moving the rail, the first doing device and the upper punch may be moved sequentially above the die.

In another embodiment, the second positioning device may be a disc device. The disc device may include a disc and an actuator configured to rotate the disc. The first dosing device and upper punch may be fixed to a lower side of the disc. In rotating the disc, the first doing device and the upper punch may be moved sequentially above the die.

Such object can be further achieved by a method for producing a specifiable number of individualized two-layer tablets with a specifiable quantity of the first active substance and a specifiable quantity of a second active substance, wherein the previously described method is carried out with the following:

inputting a second-layer target mass into the input device and transmitting the second-layer target mass to the controller, wherein the second-layer target mass indicates the quantity of the second active substance per two-layer tablet,

positioning a second dosing device of a second material container above the central opening by the second positioning device,

filling a quantity of a second active substance that corresponds to the second-layer target mass into the central opening by the second dosing device,

positioning the upper punch above the central opening by the second positioning device, and

pressing the second active substance by vertically extending the pressure face of the upper punch into the central opening,

wherein, except for inputting the second-layer target mass and transmitting the second-layer target mass, the positioning of the second dosing device, filling the quantity of the second active substance, positioning the upper punch and pressing the second active substance are carried out after pressing the first active substance and before dispensing the pressed tablet.

Before this method is performed, the second material container that is filled with the second active substance can be fixed to the tablet press such that it can be moved in a horizontal direction by the second positioning device. The first material container and/or the second material container can comprise an RFID chip. In this manner, the tablet press can clearly identify the material container no matter the position at which the particular material containers are installed in the tablet press. The measuring of the second-layer target mass can be performed just like the measuring of the target mass. Before the second active substance is added, the die and the lower punch can be placed on the weighing device, and then the total mass is measured. Pressing the first active substance can take place with a lower pressure than pressing the second active substance. In this manner, pressing the first active substance can create a cavity for the second active substance and can prevent the two tablet hemispheres from falling apart after the pressing of the second active substance.

This method can advantageously enable individualized two-layer tablets to be produced that are adapted to the patient's needs. After a first layer is produced by adding and pressing the first active substance, the second dosing device can be arranged above the central opening by the second positioning device and the central opening can be filled with the second-layer target mass of the second active substance. Then, the second active substance can be pressed by the upper punch in order to form a second layer on top of the first layer. Then, the produced two-layer tablet can be released from the die in the manner described above and transferred into the tablet container.

Such object can also be achieved by a tablet press for automatically producing a specifiable number of individualized tablets with a specifiable quantity of a first active substance, comprising a press chamber, a first material container that can be filled with the first active substance, a first dosing device, a controller, and an input device, wherein the press chamber has an extendable upper punch, an extendable lower punch, at least one die, and a weighing device arranged under the at least one die, by which a mass of a material filled into a central opening in the die is measurable by placing the die and the lower punch on the weighing device, wherein the input device is configured to transmit a target number and a target mass of the individualized tablets to be produced to the controller, and wherein the controller is configured to read out measured data from the weighing device and actuate the first dosing device, the upper punch, and the lower punch such that a quantity of the first active substance that corresponds to the target mass is filled into the central opening of the die and pressed by the upper punch and the lower punch into a tablet, and to repeat this process until the target number of tablets has been produced.

The tablet press can embody the same advantages, features, and characteristics as the previously described method for automatically producing a specifiable number of individualized tablets with a specifiable quantity of a first active substance by a tablet press.

The tablet press can advantageously represent a decentralized device for producing individualized tablets. The individualized tablets can be produced directly on site, meaning in a doctor's office, a pharmacy, or a pharmaceutical production facility by expert doctors or pharmacists, or by patients who have been trained accordingly. With the individualized tablets, incorrect dosing/doses that are too high can be avoided and taking the tablets is made easier for the patient, since they no longer have to be split.

The upper punch and/or the lower punch can be vertically extendable. According to one embodiment, the lower punch can be arranged in a fixed location in the press chamber. The position of the lower punch in the press chamber therefore does not change in a horizontal direction according to this embodiment; the lower punch can only be extended in a vertical direction and lifted or lowered. This can enable the pressing force acting on the lower punch to be measured easily.

The pressed tablet can be released by extending the lower punch in the central opening. The tablet press can comprise a receiving chamber for receiving a tablet container. The first dosing device can be formed as part of the first material container, or separate therefrom. The input device can comprise input means for entering the target number that indicates the number of individualized tablets to be produced.

According to one embodiment, the tablet press can comprise at least two dies, the central openings of which can have different inner circumferences, wherein the controller can be configured to calculate a filling volume and thus a target circumference, taking into account the target mass, and if necessary a bulk density, of the first active substance, to select a target die from the at least two dies, the inner circumference of the target die's central opening corresponding to the target circumference, and to actuate a first positioning device such that the target die is arranged above the lower punch, wherein the first positioning device can be a rail device arranged between the upper punch and the lower punch, on which the at least two dies can be arranged, such as being fixed.

The upper punch can comprise at least two vertically telescoping upper punch shafts arranged inside each other, by which an outer circumference of a pressure face of the upper punch can be adapted to the inner circumference of the central opening of the at least one die by individually extending the upper punch shafts.

Also, the lower punch can comprise at least two vertically telescoping lower punch shafts arranged inside each other, by which an outer circumference of a pressure face of the lower punch can be adapted to the inner circumference of the central opening of the at least one die by individually extending the lower punch shafts.

The press chamber can comprise a first pushing and pulling device, by which at least one upper locking pin can be pushed into a horizontal hole in side walls of the upper punch shafts to vertically fix a definable number of the upper punch shafts, wherein the press chamber can comprise a second pushing and pulling device, by which at least one lower locking pin can be pushed into a horizontal hole in side walls of the lower punch shafts to vertically fix a definable number of the lower punch shafts. The first pushing and pulling device and the second pushing and pulling device may comprise a linear actuator to push and pull the locking pin.

By the locking pins, it can be achieved that only the desired punch shafts are extended when the upper punch and the lower punch are extended.

The upper punch can be extended by an upper piston that presses on the upper punch from above. The lower punch can be correspondingly extended by a lower piston that presses on the lower punch from below. The upper piston can be connected exclusively to the innermost upper punch shaft and the lower piston can be connected exclusively to the innermost lower punch shaft. If further upper punch shafts and/or lower punch shafts are to be additionally extended, these further upper punch shafts and/or lower punch shafts can be coupled with the inner upper punch shaft and/or lower punch shaft by a coupling pin. The coupling pin can be driven by a rod-shaped pushing and pulling device. The pushing and pulling device can be guided in a groove running vertically inside a punch carrier such that it can move in sync with the vertical movement of the extended upper punch shafts and/or lower punch shafts.

The tablet press can comprise at least one lifting device that can be configured to lift and lower the at least one die and the lower punch, such as in a vertical direction.

The die and the lower punch can be placed on the weighing device by the at least one lifting device before adding the first active substance or the second active substance, and can be lifted after adding the first active substance or the second active substance.

Also, a second positioning device can be included by which the upper punch and the first dosing device, such as a second dosing device of a second material container, can alternatingly be arranged vertically above the central opening in the die arranged above the lower punch.

The second positioning device can be a rail device on which the first dosing device and the upper punch can be arranged, such as being fixed, and can be moved in a horizontal direction, wherein the first dosing device can be movably arranged on the rail device also in a vertical direction, such as being fixed.

According to an embodiment, the press chamber can be dust-tight, wherein the receiving chamber can be closed in a dust-tight manner.

Advantageously, this can avoid contamination of the receiving chamber and/or of a tablet container arranged in the receiving chamber and/or of the region outside the tablet press.

The lower punch and the die can form a unit. The lower punch and the die can be coupled to each other in a vertical direction by a spring.

The lower punch can have a recess in its lower side that is configured to form-fit with an elevation on the upper side of the weighing device.

In this manner, it is ensured that the lower punch and the die rest securely on the weighing device while filling, and that the mass of the added active substance can be exactly determined.

Further features will become evident from the description of embodiments, together with the claims and the appended drawings. Embodiments can fulfill individual features or a combination of several features.

The embodiments are described below, without restricting the general idea of the invention, based on exemplary embodiments in reference to the drawings, whereby we expressly refer to the drawings with regard to the disclosure of all details that are not explained in greater detail in the text. In the drawings:

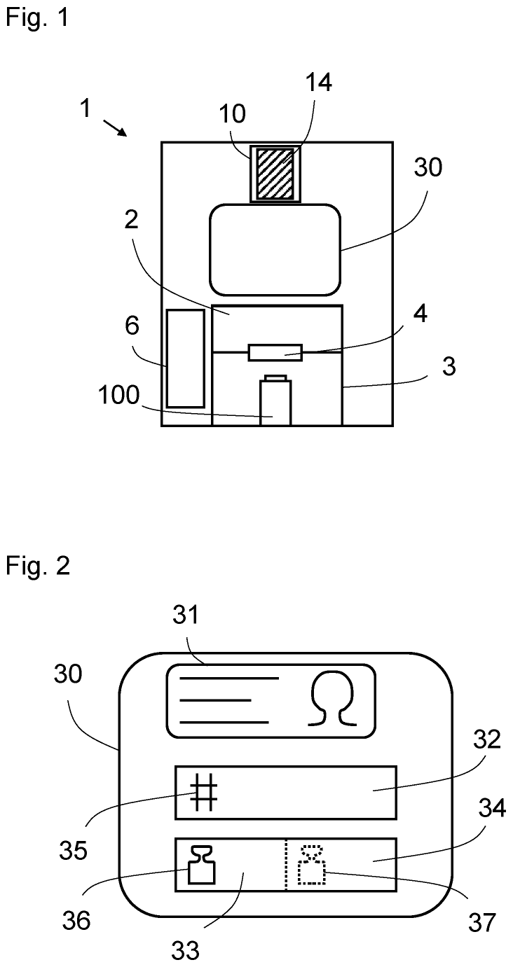

FIG. 1 illustrates a schematically simplified diagram of a tablet press for automatically producing a specifiable number of individualized tablets with a specifiable quantity of a first active substance,

FIG. 2 illustrates a schematically simplified diagram of an input device of the tablet press from FIG. 1,

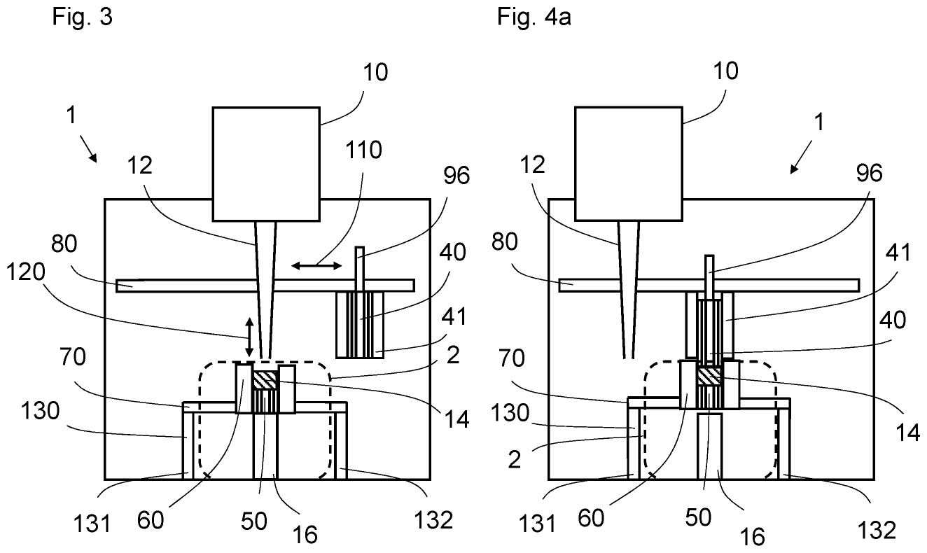

FIG. 3 illustrates a schematically simplified cross-sectional view of a press chamber and a material container of a tablet press while adding an active substance,

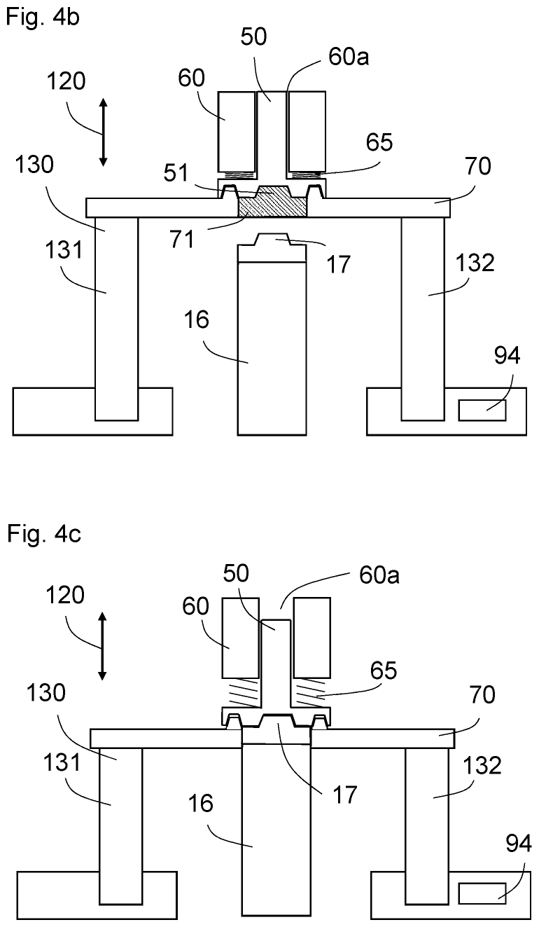

FIG. 4a illustrates a schematically simplified cross-sectional view of a press chamber and a material container of a tablet press during pressing of an active substance,

FIG. 4b illustrates a schematically simplified cross-sectional view of a lower punch and a die which are coupled by a spring, and a weighing device,

FIG. 4c illustrates a schematically simplified cross-sectional view of the lower punch of the die, and the weighing device of FIG. 4b while filling,

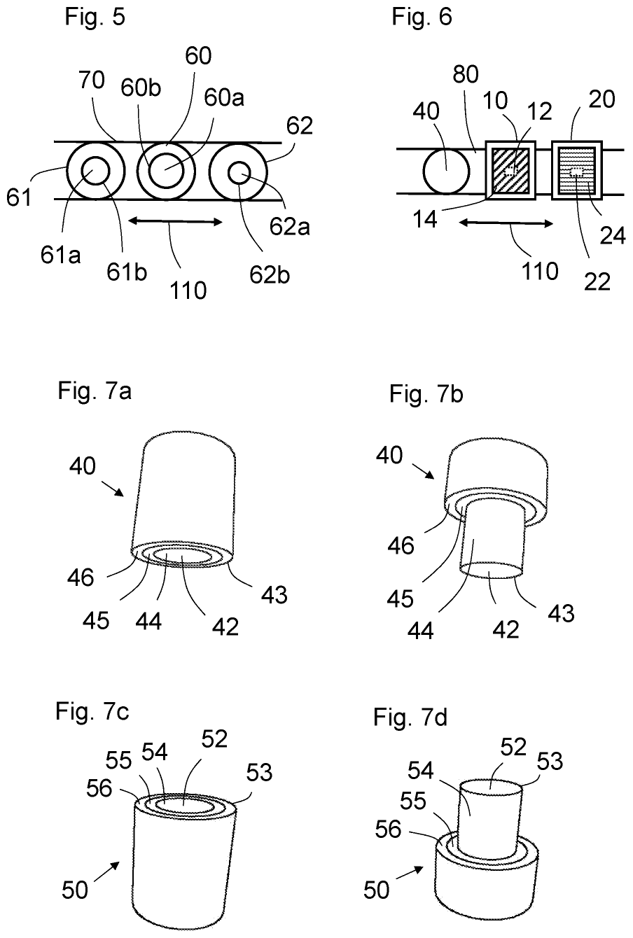

FIG. 5 illustrates a schematically simplified plan view of a first positioning device,

FIG. 6 illustrates a schematically simplified plan view of a second positioning device,

FIG. 7a-d illustrate schematically simplified diagrams of an upper punch with telescoping upper punch shafts arranged inside each other and a lower punch with telescoping lower punch shafts arranged inside each other,

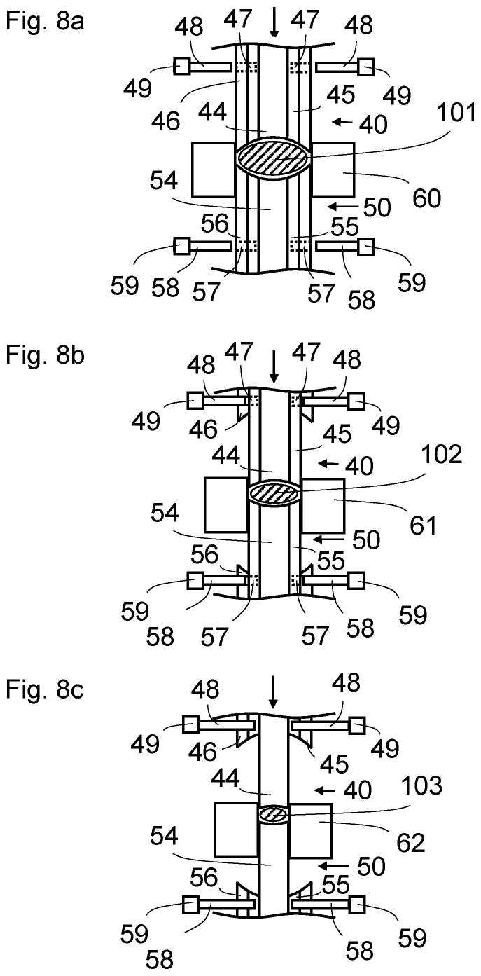

FIG. 8a-c illustrate schematically simplified cross-sectional views of an upper punch, a lower punch, and multiple dies during the pressing process of tablets with different active substance quantities,

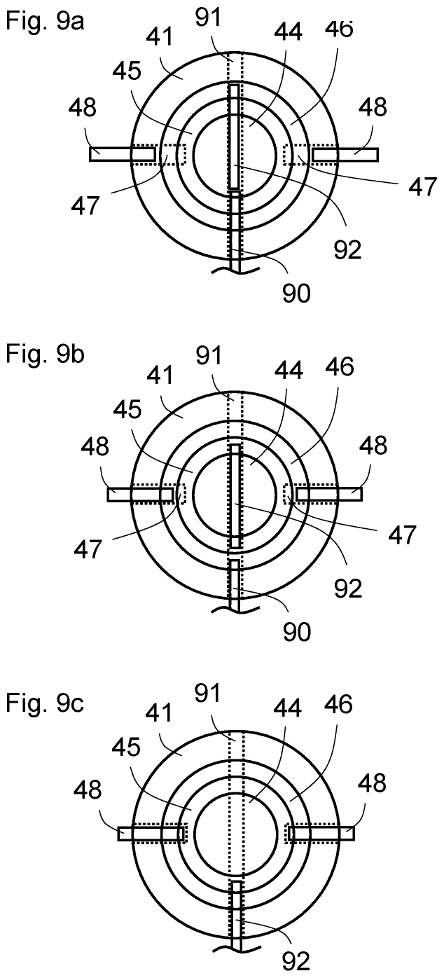

FIG. 9a-c illustrate schematically simplified plan views of an upper punch during the pressing process of tablets with different active substance quantities.

In the drawings, the same or similar elements and/or parts are, in each case, provided with the same reference numerals such that they are not introduced again in each case.

### DETAILED DESCRIPTION

FIG. 1 shows a schematically simplified exemplary embodiment of a tablet press 1. The tablet press 1 comprises a press chamber 2, a first material container 10 that is filled with a first active substance 14, a receiving chamber 3 in which a tablet container 100 can be arranged, a transfer device 4 for transporting the produced tablets from the press chamber 2 into the tablet container 100, a control system 6 for controlling the tablet press 1, and an input device 30 for operating the tablet press 1. In the embodiment of the tablet press 1 shown in FIG. 1, the first material container 10 is arranged in a housing of the tablet press 1 and is detachably fixed there. For example, the first material container 10 can be removed from the housing through a flap, which is not shown, in order to replace or refill it. The input device 30 can comprise a security access system. For example, the input device 30 is secured by an access code or comprises a fingerprint scanner.

According to one embodiment, the press chamber 2 is configured as an exchangeable press chamber insert. For this purpose, the press chamber 2 is removably fixed in the tablet press 1. To be exchanged, the press chamber insert can be removed from the tablet press and replaced by another press chamber insert.

The control system 6 is, for example, a computer, controller, processor, circuit(s), CPU, etc. that is configured to control the tablet press 1 such that a target number of individualized tablets with a target mass of the first active substance 14 is produced automatically. The receiving chamber 3 can be closed according to an embodiment such that no dust escapes from the tablet press 1 and no contaminants enter the tablet press 1 during the production of the tablets.

FIG. 2 shows a schematically simplified detailed view of the input device 30 which is a touchscreen on the tablet press 1 in the shown embodiment. The input device 30 comprises a data display 31, for example a region of the screen, on which information about the first active substance 14 and/or a patient is displayed. The input device 30 further comprises multiple input means 32, 33, 34, which are, for example, additional regions of the screen. By the input means 32, 33, 34, the target number 35, the target mass 36, and if necessary, a second-layer target mass 37 can be input. The input values are displayed for verification. In addition, a further input means (not shown) is provided, with which the production of the tablets can be started. According to another embodiment, the input device 30 is an external unit or an interface for communicating with an external unit. In this case, the target number 35, the target mass 36 and, if applicable, the second layer target mass 37 are entered in the external unit and transmitted to the control system 6.

In FIG. 3, a schematically simplified cross-sectional view of an embodiment of the tablet press 1 with the press chamber 2 and the first material container 10 is shown. The first material container 10 comprises a first dosing device 12 for filling the first active substance 14 into the central opening of a die 60. According to another embodiment, the first material container 10 is connected to the first dosing device 12. The first dosing device 12 is, for example, a closable funnel.

A lower punch 50 is arranged in the press chamber 2 such that it extends from below into the die 60. The lower punch 50 can be extended in a vertical direction. Before the first active substance 14 is filled into the die 60, the lower punch 50 is extended into the die 60 from below such that a trough for receiving the first active substance 14 is created. Alternatively, the lower punch 50 is permanently arranged in the die 60. In both cases, the lower punch 50 can be moved in a vertical direction 120 in the central opening in the die.

The die 60 and/or the lower punch 50 are arranged on a platform 70. According to one embodiment, the platform 70 is a first positioning device. Additional dies (not shown in FIG. 3) are fixed to this first positioning device and can be moved into a position above the lower punch 50 by rotating the first positioning device. According to an alternative embodiment, only one die 60 and one lower punch 50 are affixed to the platform 70.

The platform 70 and therefore the die 60 and the lower punch 50 as well can be raised and lowered in a vertical direction 120 by a lifting device 130 that comprises a first spindle 131 and a second spindle 132. Moreover, the pressure acting on the lower punch 50 during the pressing process can be determined by a pressure measuring device (not shown) on the lifting device 130 and/or by the power consumption of the lifting device 130. A weighing device 16 is arranged below the die 60 and the lower punch 50.

An upper punch 40 surrounded and supported by an upper punch carrier 41 is also arranged in the tablet press 1. The upper punch 40 can be extended in the vertical direction by an upper piston 96.

The first dosing device 12 and the upper punch 40 are fixed to a second positioning device 80, which is a rail device in the shown embodiment.

To fill the die 60 with the first active substance 14, the second positioning device 80 is actuated by the control system 6 such that the first dosing device 12 is arranged above the lower punch 50 and the die 60, as shown in FIG. 3. Naturally, this only occurs if the first dosing device 12 is not yet arranged above the lower punch 50. Moreover, the die 60 and the lower punch 50 are placed on the weighing device 16 so that, by the weighing device 16, the overall weight of the die 60, the lower punch 50, as well as the first active substance 14 filled into the die are measured. To prevent the first active substance 14 from spilling while being added, the first dosing device 12 is lowered so that the bottom end of the first dosing device 12 is below the upper edge of the die 60. Then, the central opening of the die 60 is filled with the first active substance 14 by the first dosing device 12 until the target mass 36 is reached. This is monitored with the weighing device 16.

After the die 60 is filled, the first active substance 14 is pressed. This is shown in FIG. 4a. For this purpose, the die 60 and the lower punch 60 are lifted off the weighing device 16 by the lifting device 130, the dosing device 12 is moved vertically upward, and the second positioning device 80 is controlled by the control system 6 so that the upper punch 40 is arranged above the lower punch 50 and the die 60. Then, the upper punch 40 is extended into the central opening of the die 60 from above by the upper piston 96. In this manner, the first active substance 14 is pressed between the pressure faces of the lower punch 50 and the upper punch 40 in order to form a tablet.

After pressing the tablet, both the upper punch 40 and the lower punch 50 move upwards to release the tablet from the die 60. Then, the tablet is pushed down from the lower punch 50, for example by a pushing device (not shown) of the transfer device 4, such that it falls through a channel of the transfer device 4 into the tablet container 100.

This method is repeated until the target number 35 of tablets has been produced. After that, the receiving chamber 3 can be opened and the tablet container 100 removed. The tablet container 100 is now filled with the target number 35 of individualized tablets with the target mass 36 of the first active substance 14.

FIG. 4b shows a schematically simplified cross-sectional view of an exemplary embodiment of a weighing device 16, a die 60, and a lower punch 50. In the shown embodiment, only one die 60 is included that forms a unit together with the lower punch 50, and they are coupled to each other by a spring 65. In FIG. 4b, the spring 65 is compressed, whereby the lower punch 50 extends to the upper edge of the central opening 60a of the die. This is for example recommendable while ejecting a pressed tablet. The unit consisting of the lower punch 50 and the die 60 is arranged on the platform 70 that can be moved in a vertical direction 120 by the lifting device 130. The weighing device 16 is arranged below the die 60 and the lower punch 50. On its upper side, the weighing device 15 has an elevation 17 that is configured to form-fit with a recess 51 in the lower side of the lower punch 50. The recess 51 of the lower punch 51 is adjacent to a recess 71 in the platform 70 that together form a cavity which is shown with a dashed line in FIG. 4b. With the recess 71, the weighing device 16 can move through the platform 70 so that it comes into contact with the lower punch. The lifting device 130 also comprises a pressure measuring device 94. This pressure measuring 94 device is for example a piezoelectric load cell, a pneumatic load cell or a hydraulic load cell. With the pressure measuring 94, the pressure acting on the punches can be measured during the pressing process.

FIG. 4c shows the system from FIG. 4b while adding an active substance. The die 60 and the lower punch 50 are pulled apart in a vertical direction 120 under the spring force of the spring 65 which produces a trough in the central opening 60a of the die 60 into which the active substance is filled. The lower punch 50 lies on the weighing device 16 so that the elevation 17 engages in a form-fit in the seat 51. The platform 70 is lowered until the lower punch 50 lies exclusively on the weighing device 16 and not on the platform 70. In this way, the weighing device 16 measures the total mass of the lower punch 50, the die 60, and the active substance filled into the trough while filling. The active substance is added as usual by the dosing device 12 which is not shown in FIGS. 4b and 4c.

FIG. 5 shows a schematically simplified top view of a platform 70 configured as a first positioning device according to a possible embodiment of the tablet press 1. In the shown embodiment, the first positioning device is configured as a rail device. Three dies 60, 61, 62 are fixed to the first positioning device. The dies 60, 61, 62 each have a central opening 60a, 61a, 62a with different circular inner circumferences 60b, 61b, 62b. The central openings 60a, 61a, 62a pass through the platform 70 such that the punches 40, 50 are not blocked by the platform 70. Which of the dies 60, 61, 62 is arranged above the lower punch 50 can be set by translating the first positioning device in a horizontal direction 110. In this manner, a target circumference of the produced tablets can be set such that tablets with a wide range of target masses 36 of the first active substance 14 can be produced of the tablet press 1.

Alternatively, oval or other shapes can be provided instead of the circular shape of the central openings 60a, 61a, 62a, enabling the shape of the tablets to be adjusted.

FIG. 6 shows a schematically simplified plan view of the second positioning device 80. In the shown embodiment, this is configured as a rail device. The upper punch 40, the first material container 10 and, in the shown embodiment, a second material container 20 are fixed to the rail device and can be alternately arranged above the lower punch 50 by being translated in a horizontal direction 110 by the rail device.

The second material container 20 is filled with a second active substance 24 for producing a two-layer tablet. For this purpose, e.g., after the pressing of the first active substance 14 by the second positioning device 80, the second dosing device 22 of the second material container 20 is arranged above the lower punch 50 and the central opening 60a of the die 60 arranged thereon is filled with the second active substance 24. Then, the upper punch 40 is moved above the lower punch 50 and the two-layer tablet is pressed. To produce a two-layer tablet with adjacent layers, a first corner of the die 60 is filled with the first active substance 14 by the first dosing device 12, and a second corner of the die 60 is filled with the second active substance 24 by the second dosing device, and the tablet is then pressed. As the dosing devices 12, 22 are arranged below the material containers 10, 20, the dosing devices 12, 22 would normally not be visible in FIG. 6 and are thus drawn with dashed lines.

FIGS. 7a and 7b show an embodiment of a telescoping upper punch 40. The upper punch 40 comprises three telescoping upper punch shafts 44, 45, 46 arranged inside each other. In FIG. 7a, all three upper punch shafts 44, 45, 46 are extended together such that the outer circumference 43 of the pressure face 42 of the upper punch 40 corresponds to the outer circumference of the outermost upper punch shaft 46. In FIG. 7b, exclusively the innermost upper punch shaft 44 is extended such that the outer circumference 43 of the pressure face 42 of the upper punch 40 corresponds to the outer circumference of the innermost upper punch shaft 44. The outer circumferences of the upper punch shafts 44, 45, 46 are each complementary in shape to the inner circumferences 60b, 61b, 62b of the dies 60, 61, 62.

FIGS. 7c and 7d show an embodiment of a telescoping lower punch 50 that is formed as a mirror image to the upper punch 40. Just as with the upper punch 40, the outer circumference 53 of the pressure face 52 of the lower punch 50 can be varied by individually extending the lower punch shafts 54, 55, 56.

In FIG. 8a, a schematically simplified cross-sectional view of a telescoping upper punch 40, a telescoping lower punch 50, and a die 60 are shown during the pressing of a large tablet 101. In this case, the die 60 with the large central opening 60a is used. To press the tablet 101, all upper punch shafts 44, 45, 46 and lower punch shafts 54, 55, 56 are used. In the shown embodiment, one or more horizontal holes 47 are provided in the upper punch shafts 44, 45, 46, into which holes upper locking pins 48 can be pushed by one or more schematically indicated first pushing and pulling devices 49. Analogously, in the shown embodiment, one or more horizontal holes 57 are provided in the lower punch shafts 54, 55, 56, into which holes lower locking pins 58 can be pushed by one or more schematically indicated second pushing and pulling devices 59. Since the largest die 60 is used, the locking pins 48, 58 are retracted such that they do not engage in the horizontal holes 47, 57. The pushing direction of the upper piston 96 is represented by an arrow.

FIG. 8b shows a schematically simplified cross-sectional view during the production of a tablet 102 with a medium size. For this purpose, the die 61 is used that has the central opening 61a with the medium size. The locking pins 48, 58 are partially inserted into the holes 47, 57 such that the outermost punch shafts 46, 56 are vertically fixed and only the middle and the inner punch shafts 44, 45, 54, 55 are extended. In this manner, the outer circumferences 43, 53, of the pressure faces 42, 52 are adapted to the inner circumference 61b of the die 61.

FIG. 8c shows a schematically simplified cross-sectional view during the production of a small tablet 103. For this purpose, the die 62 is used that has the smallest central opening 62a. The locking pins 48, 58 are fully inserted into the holes 47, 57 such that the outermost and the middle punch shafts 45, 46, 55, 56 are vertically fixed and only the inner punch shafts 44, 45, 54, 55 are extended. In this manner, the outer circumferences 43, 53, of the pressure faces 42, 52 are adapted to the inner circumference 62b of the die 62.

FIG. 9a shows a schematically simplified plan view of FIG. 8a with an additional representation of the upper punch carrier 41 as the outermost ring. A further horizontal hole 91 is provided perpendicularly to the holes 47. A coupling pin 92 is guided in the hole 91 and is inserted into and retracted from the hole 91 by a third pushing and pulling device 90.

In the shown embodiment in FIG. 8a, 8b, 8c, 9a, 9b, 9c, the upper piston 96 directly drives exclusively the inner upper punch shaft 44, while a lower piston (not shown) directly drives exclusively the inner lower punch shaft 54. By the coupling pin 92, the upper punch shafts 44, 45, 46 are coupled with each other, if necessary, such that they are moved up and down synchronously. This is provided analogously for the lower punch 50. In FIG. 9a, the coupling pin 92 couples all three upper punch shafts 44, 45, 46 with each other such that they are vertically extended together. A vertical groove is provided in the upper punch carrier 41 so that the third pushing and pulling device 90 does not block in the upper punch carrier 41.

FIG. 9b shows a schematically simplified plan view of FIG. 8b. The coupling pin 92 couples only the inner upper punch shaft 44 with the middle upper punch shaft 45, while the outer upper punch shaft 46 is vertically fixed.

FIG. 9c shows a schematically simplified plan view of FIG. 8c. The coupling pin 92 is inserted such that only the inner upper punch shaft 44 is extended, while the middle upper punch shaft 45 and the outer upper punch shaft 46 are vertically fixed.

While there has been shown and described what is considered to be preferred embodiments, it will, of course, be understood that various modifications and changes in form or detail could readily be made without departing from the spirit of the invention. It is therefore intended that the invention be not limited to the exact forms described and illustrated, but should be constructed to cover all modifications that may fall within the scope of the appended claims.

### LIST OF REFERENCE SIGNS

1 Tablet press
2 Press chamber
3 Receiving chamber
4 Transfer device
6 Control system
10 First material container
12 First dosing device
14 First active substance
16 Weighing device
17 Elevation
20 Second material container
22 Second dosing device
24 Second active substance
30 Input device
31 Data display
32, 33, 34 Input means
35 Target number
36 Target mass
37 Second-layer target mass
40 Upper punch
41 Upper punch carrier
42 Pressure face
43 Outer circumference
44, 45, 46 Upper punch shafts
47 Horizontal hole
48 Upper locking pin
49 First pushing and pulling device
50 Lower punch
51 Recess
52 Pressure face
53 Outer circumference
54, 55, 56 Lower punch shafts
57 Horizontal hole
58 Lower locking pin
59 Second pushing and pulling device
60, 61, 62 Die
60a, 61a, 62a Central opening
60b, 61b, 62b Inner circumference
65 Spring
70 Platform
71 Recess
80 Second positioning device
90 Third pushing and pulling device
91 Horizontal hole
92 Coupling pin
94 Pressure measuring device
96 Upper piston
100 Tablet container
101, 102, 103 Tablet
110 Horizontal direction
120 Vertical direction
130 Lifting device
131 First spindle
132 Second spindle

What is claimed is:

1. A method for automatically producing a number of individualized tablets with a first quantity of a first active substance by a tablet press, the method comprising:
inputting a target number which indicates the number of tablets to be produced and a first target mass which indicates the quantity of the first active substance per tablet,
transmitting the target number and the first target mass to a controller of the tablet press, wherein the controller is configured to control a first dosing device, a weighing device, an extendable upper punch, and an extendable lower punch,
filling a quantity of the first active substance which corresponds to the first target mass from a first material container into a central opening of a die by the first dosing device, wherein while filling, a pressure face of the lower punch closes the central opening on one side, and the amount of the first active substance filled into the central opening is measured by the weighing device on which the lower punch and the die are placed while filling,
pressing the first active substance by extending a pressure face of the upper punch into the central opening,
releasing a pressed tablet from the die by extending the pressure face of the lower punch into the central opening, and
repeating the filling of the quantity of the first active substance, pressing of the first active substance, and releasing of the pressed tablet until a number of pressed tablets corresponds to the target number.

2. The method according to claim 1, wherein the outer circumference of the pressure face of the upper punch for pressing the first active substance is adapted to the inner circumference of the die in that a number of vertically telescoping upper punch shafts of the upper punch arranged inside each other are extended downwards, wherein the outer circumference of the outer extended upper punch shaft corresponds to the inner circumference of the die.

3. The method according to claim 1, wherein the outer circumference of the pressure face of the lower punch is adapted to the inner circumference of the die before the filling of the quantity of the first active substance in that a number of vertically telescoping lower punch shafts of the lower punch arranged inside each other are extended upwards, wherein the outer circumference of the outer extended lower punch shaft corresponds to the inner circumference of the die.

4. The method according to claim 1, wherein a mass of the first active substance filled into the central opening is measured by placing the die and the lower punch on the weighing device before filling wherein the weighing device measures the total mass acting on it while filling, which comprises the mass of the die, the mass of the lower punch, and the mass of the first active substance filled into the central opening, wherein the die and the lower punch are lifted after filling and before pressing the first active substance.

5. The method according to claim 4, wherein
the die and the lower punch are placed on the weighing device before filling by at least one lifting device, and
the die and the lower punch are lifted after filling and before pressing the first active substance by the at least one lifting device.

6. The method according to claim 5, wherein the pressure acting on one or more of the lower punch and the upper punch is measured at the at least one lifting device.

7. The method according to claim 6, wherein the pressure is measured by a pressure measuring device.

8. The method according to claim 1, wherein the first dosing device is arranged vertically above the central opening by a positioning device before adding the quantity of the first active substance, wherein the upper punch is arranged vertically above the central opening by the positioning device after adding the quantity of the first active substance and before pressing the first active substance, wherein the first dosing device is also arranged.

9. The method according to claim 8, wherein
the positioning device is a rail device that can be moved in a horizontal direction, and
the first dosing device is fixed on the positioning device so as to be movable in the vertical direction.

10. The method according to claim 1, further comprising subsequent to the pressing of the first active substance and prior to the releasing of the pressed tablet:
inputting a second-layer target mass and transmitting the second-layer target mass to the controller, wherein the second-layer target mass indicates a quantity of a second active substance per tablet,
positioning a second dosing device of a second material container above the central opening by a positioning device,
filling a second quantity of the second active substance that corresponds to a second-layer target mass into the central opening by the second dosing device,
positioning the upper punch above the central opening by the positioning device, and
pressing the second active substance by extending the pressure face of the upper punch into the central opening,
wherein, the repeating comprises repeating the filling of the quantity of the first active substance, pressing of the first active substance, filling the second quantity of the second active substance pressing the second active substance and releasing of the pressed tablet until the number of pressed tablets corresponds to the target number.

11. A tablet press for automatically producing a specifiable number of individualized tablets with a specifiable quantity of a first active substance, the tablet press comprising:
a press chamber,
a first material container configured to be filled with the first active substance,
a first dosing device,
a controller, and
an input device,
wherein the press chamber has an extendable upper punch, an extendable lower punch, at least one die, and a weighing device arranged under the at least one die, by which a mass of a material filled into a central opening in the die is measurable by placing the die and the lower punch on the weighing device,
the input device is configured to transmit a target number and a target mass of the individualized tablets to be produced to the controller, and
the controller is configured to read out measured data from the weighing device and actuate the first dosing device, the upper punch, and the lower punch such that a quantity of the first active substance that corresponds to the target mass is filled into the central opening of the die and pressed by the upper punch and the lower punch into a tablet, and to repeat the pressing until a target number of tablets has been produced.

12. The tablet press according to claim 8, wherein the upper punch comprises at least two vertically telescoping upper punch shafts arranged inside each other, by which an outer circumference of a pressure face of the upper punch can be adapted to the inner circumference of the central opening of the at least one die by individually extending the upper punch shafts.

13. The tablet press according to claim 12, wherein the lower punch comprises at least two vertically telescoping lower punch shafts arranged inside each other, by which an outer circumference of a pressure face of the lower punch can be adapted to the inner circumference of the central opening of the at least one die by individually extending the lower punch shafts.

14. The tablet press according to claim 13, wherein the press chamber comprises a first pushing and pulling device, by which at least one upper locking pin can be pushed into a horizontal hole in side walls of the upper punch shafts to vertically fix a definable number of the upper punch shafts, wherein the press chamber comprises a second pushing and pulling device, by which at least one lower locking pin is pushed into a horizontal hole in side walls of the lower punch shafts to vertically fix a definable number of the lower punch shafts.

15. The tablet press according to claim 11, wherein the tablet press further comprises at least one lifting device that is configured to lift and lower the at least one die and the lower punch.

16. The tablet press according to claim 15, wherein the at least one lifting device is configured to lift and lower the at least one die and the lower punch in a vertical direction (120).

17. The tablet press according to claim 11, further comprising a positioning device by which the upper punch and the first dosing device can alternatingly be arranged vertically above the central opening in the die arranged above the lower punch.

18. The tablet press according to claim 17, wherein the positioning device further arranges a second dosing device of a second material container above the central opening in the die.

19. The tablet press according to claim 17, wherein the positioning device is a rail device on which the first dosing device and the upper punch are arranged and can be moved in a horizontal direction, wherein the first dosing device is movably arranged on the rail device in a vertical direction.

20. The tablet press according to claim 11, wherein the lower punch has a recess in its lower side that is configured to form-fit with an elevation on an upper side of the weighing device.
